(19) United States
(12) Patent Application Publication
(10) Pub. No.: US 2025/0118408 A1
(43) Pub. Date: Apr. 10, 2025

(54) Method and equipment for improving cardiac arrest resuscitation (Pocket Crash Cart)

(71) Applicants: Tamara Aliza Freeman, Litchfield, NH (US); Michelle Alyssa Freeman, Litchfield, NH (US)

(72) Inventors: Tamara Aliza Freeman, Litchfield, NH (US); Michelle Alyssa Freeman, Litchfield, NH (US)

(73) Assignee: Tamara Aliza Freeman, Litchfield, NH (US)

(21) Appl. No.: 18/377,005

(22) Filed: Oct. 5, 2023

(51) Int. Cl. G16H 20/17; A61B 5/363

(52) U.S. Cl. CPC G16H 20/17; A61B 5/363

(57) ABSTRACT

Method and equipment for improving cardiac arrest resuscitation (Pocket Crash Cart) includes:
1. Manual or Automatic Pocket Crash Cart which attaches to the patient via IV/OS to allows quick and accurate administration of emergency medication manually or remotely.
2. The software for mirroring display screen of manual defibrillator or AED/computer/laptop/phone and video cameras allowing to transfer patient information from the Cardiac Arrest scene especially in hard-to-reach locations (cruise ship, expeditions) to CMF via internet for expert advice and/or administration of emergency medication in the absence of ASLC provider.

### BACKGROUND OF THE INVENTION

### 1. Field of Invention

This invention relates generally to the resuscitation process during sudden Cardiac Arrest (CA), more specifically to CA in rural or hard to reach locations. This method would aid emergency responders in administering emergency medication.

Cardiac arrest occurs when the heart's electrical transmission system malfunctions due to underlying diseases such as heart attack, heart failure, arrhythmias, cardiomyopathy, ventricular fibrillation, and prolong Q-T syndrome. As a result, the heart stops beating suddenly, which stops blood flow to a person's organs. This can cause a person to stop breathing and lose consciousness, leading to disability or death if not treated immediately.

Sudden Cardiac Arrest can happen to any person in any place. Hence, there is a need for a tool that can allow Emergency Medical Services (EMS) personnel to quickly administer appropriate medication to avoid delays in treatment.

### DESCRIPTION OF THE PRIOR ART

The current practice for treating CA outside of a hospital setting is for responders to first assess the collapsed individual for absence of consciousness, heartbeat, and/or breathing. Then, responders can call 911 to call EMS to the scene. Then, initial responders can start cardiopulmonary resuscitation (CPR), which can include chest compression, rescue breathing, and using a defibrillator. Defibrillators can be automated (AED) or manual (md), and these devices analyze/display heart rhythms and identify if electric shock is suggested. Once EMS providers arrive, they are equipped and trained to provide CPR. Some are Advance Cardiac Life Support (ACLS) certified, which allows them to administer emergency cardiac medications.

The primary problem with conventional CA diagnostic tools and medication administration is the time it takes for appropriately trained EMS personnel to arrive at the scene of the CA and go through the appropriate life-support steps.

After arriving at the scene and confirming that the person has no pulse and/or isn't breathing, EMS personnel will continue CPR. EMS personnel will also establish intravenous (IV) or intraosseous (IO) access on the patient and connect them to oxygen. Then, if an ACLS certified provider is present, they will retrieve a crash cart (a portable cart that contains all necessary equipment and medications to help with the resuscitation process) from the ambulance to administer emergency medication.

EMS teams may be equipped with a manual defibrillator (md), which gives more options to properly treat CA by displaying the patient's cardiac rhythm on the screen and allowing manual adjustments of how many Joules are delivered during the electrical shock that restarts the heart's electrical system.

An important stipulation to this response plan is that ONLY medical doctors (MD) or ACLS personnel can identify cardiac rhythms on the manual defibrillator and administer appropriate medication. This responder's tasks include:
Opening the appropriate drawer in the crash cart
Locating and opening the vial with the appropriate medication
Withdrawing the appropriate amount of medicine into a syringe
Attaching the syringe to the patient's IV/IO access
Administering the medication and flushing with Normal Saline

This process is repeated for every medication that needs to be administered. For this process to take place, it requires an ACLS provider or the MD to be present and concentrated ONLY on this role.

Through this conventional treatment method, in-hospital CA has a survival rate of around 24%, while out-of-hospital CA survival rate is around only 6%.

In this respect, the proposed method and equipment for improving cardiac arrest resuscitation departs substantially from conventional methods of CA resuscitation process and compositions of the prior art. In doing so, the proposed method provides a quick and accurate assessment of various CA scenarios and provides treatment to increase survival rate and decrease complications to patients with CA.

### SUMMARY OF THE INVENTION

In contrast to the known methods of CA resuscitation described in the prior art, the present invention improves medical intervention during medical emergencies/events by ensuring prompt medicine delivery and improving the speed at which care may be given.

The general purpose of the present invention is to provide an improved emergency treatment for cardiac arrests that has many novel features and functions, which is not anticipated, rendered obvious by any of the prior art methods either alone or in any combination thereof.

As described in the Prior Art section, it is an extremely time-consuming process to administer medication using a crash cart. This invention addresses this by containing the medication in a compact device that is convenient to attach to the patient and allows for quick and accurate adjustments in administration.

The secondary aim of this invention is to provide a communication tool and allow EMS responders without ACLS training to better help patients. To attain this, the present invention generally comprises the process of EMS collecting data at the accident site and transferring these findings to a central medical facility through a communication system. Then, an MD or ACLS provider will analyze the data, determine the necessary actions, and communicate the results to the EMS on site. Based upon the displayed results, first responders or EMS team can then make an informed decision regarding the proper treatment of the patient.

The present invention allows for health care providers around the world to have instant access to highly qualified cardiologists, MD or ACLS personnel and start treating CA with medical advice as quickly as possible.

Each computer used by EMS personnel or in the hospital would be able to communicate with the central medical facility using this invention, thereby allowing health care providers at all levels to provide accurate patient assessment and care.

There are additional features of the invention that will be described hereinafter and that will form the subject matter of the claims appended hereto.

In this respect, before explaining the invention in detail, it is to be understood that the invention is not limited in its application to the details of construction and to the arrangements of the components set forth in the following description or illustrated in drawings. The invention is capable of other embodiments and of being practiced and carried out in various ways. Also, it is to be understood that the phraseology and terminology employed herein are for the purpose of the description and should not be regarded as limited.

The primary object of the present invention is to provide quick and accurate administration of medication that may provide life-saving treatment to patients.

The secondary object of the present invention is to provide a CA resuscitation system that will overcome the shortcomings of the prior art.

Another object is to provide a system that provides a quick and accurate assessment of the various CA scenarios and accurate heart rhythm identification.

An additional object is to provide a resuscitation system that may be utilized by health care providers in various locations throughout the world.

The further object is to provide a resuscitation system that utilizes central, secondary, or tertiary health care facility providers to participate in CA.

Another object is to provide a CA diagnostic system that increases the accuracy of the medical diagnosis of the patients.

An additional object is to provide a CA resuscitation system that provides a diagnosis based upon data collected immediately at the emergency site.

A further object is to provide a CA resuscitation system that instantly displays the diagnostic results and clearly communicates between the EMS team and MD/ACLS provider at the central facility.

Another object is to provide a remotely CA resuscitation medical treatment at the site by MD/ACLS provider from the central facility utilizing a communication system.

Other objects and advantages of the present invention will become obvious to the reader, and it is intended that these objects and advantages are within the scope of the present invention.

To the accomplishment of the above and related objects, this invention may be embodied in the form illustrated in the accompanying drawings, attention being called to the fact, however, that the drawings are illustrative only, and that changes may be made in the specific use illustrated and described within the scope of the appended claims.

### BRIEF DESCRIPTION OF THE DRAWINGS

FIG. 1 is a diagram describing an improved method of cardiac resuscitation using the Pocket Crash Cart (PCC) outside of hospital settings (in hospital: Code Blue team instead of EMS arrive).

FIG. 2 is a manual Pocket Crash Cart containing multi-port IV cannulas with attached color-coded prefilled syringes with medication.

FIG. 3 is a manual defibrillator (md) with a video camera, microphone, speech recognition software, software to mirror the monitor screen, and display for notes.

FIG. 4 is a medical/911 center with MD/ACLS personnel trained in cardiac resuscitation, monitoring cardiac arrest events via camera and or mirroring AED/md screen.

FIG. 5 is automatic, remote activated medication delivery device (PCC) diagram consisting of delivery mechanism, battery, software chips, and vials with cardiac medications.

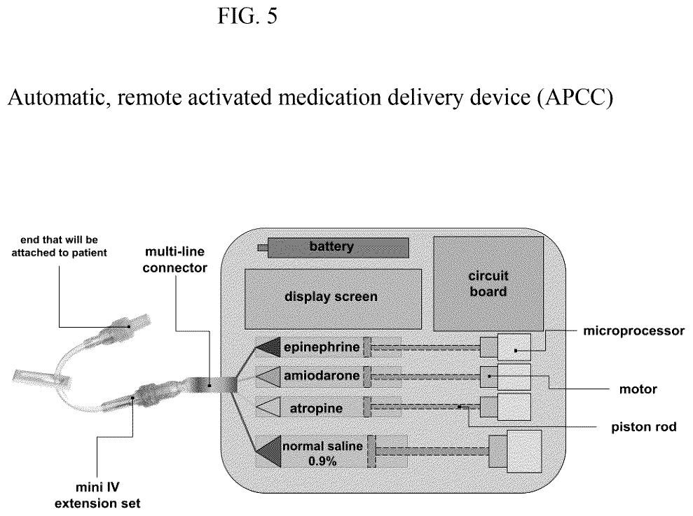

### DETAILED DESCRIPTION OF INVENTION

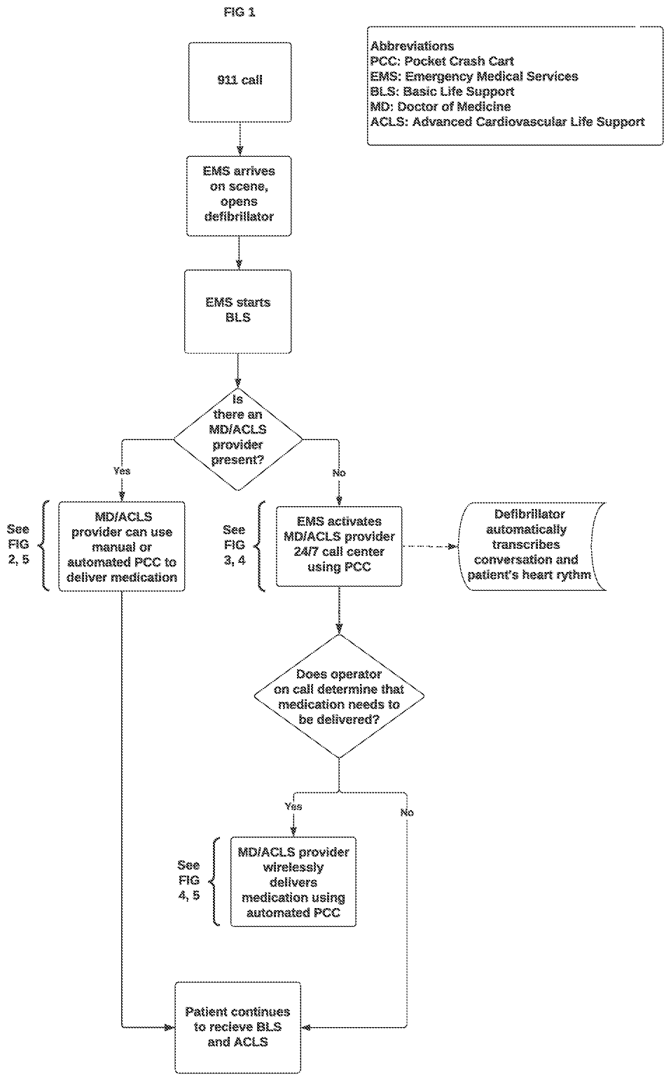

FIG. 1 explains in detail the improved method of treating CA outside of a hospital setting. When an individual collapses, the first responder starts CPR and calls a 911 center, which dispatches an EMS team.

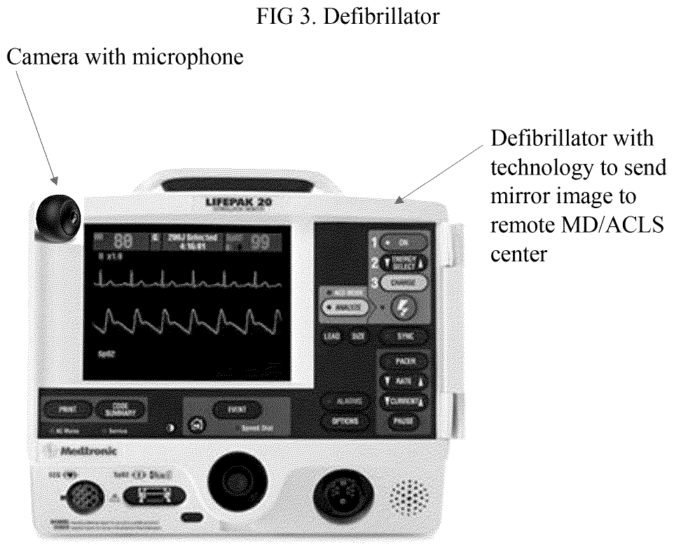

EMS team follows Basic Life Support (BLS) protocol, assessing the patient for breathing and heartbeat, providing CPR, inserting intravenous catheter (IV) or OS, and applying the oxygen and AED/md pads (FIG. 3). Turning on the defibrillator will immediately connect the EMS team via Internet to a Central Medical Facility (CMF), alerting a MD/ACLS provider that a CA is suspected.

Manual defibrillators (md) can be equipped with video cameras that have speech recognition microphones, mirroring software to record the cardiac arrest event (FIG. 3) and communicate with CMF.

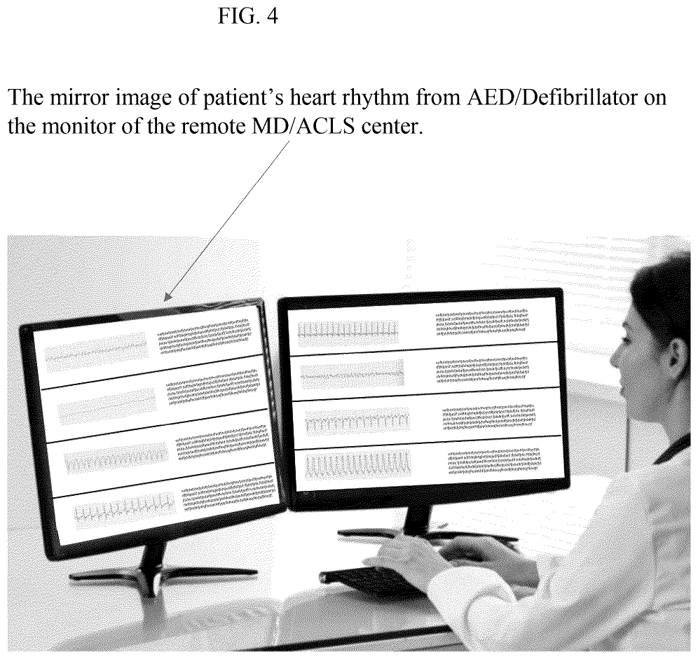

The camera with microphone can be attached to the EMS personnel or be placed on the scene, but its position must provide a very good angle of the defibrillator so that the cardiac rhythm can be identified by the MD/ACLS provider remotely. This is why the camera (or software for mirroring defibrillator display to the computer screen on the EMS device and on the medical center device (laptop, tablet, computer, etc.)) on the defibrillator is preferred, so it will send the image directly from their screen and the two-way messages can be displayed in one place (FIG. 4).

The AED can be equipped with a video camera to record the event and communicate with CMF, which would allow quick assessment by MD/ASLC provider. The video camera would not only record the events of the cardiac arrest, but also transcribe spoken words to the typed log (which now means you don't need the “recorder” on your ACLS team). These notes can be reviewed on the defibrillator monitor or on the computer screen in a CMF by the Team Leader (MD or ACLS provider) in FIG. 4.

As soon as the video camera on the defibrillator or AED turns on, the microphone will also turn on and sync to the computer (or another device such as a laptop, tablet, phone, etc.).

At the site of the accident, examples of data that can be collected are cardiac rhythm, oxygen saturation, communication, and brief history from witnesses or description of the scene. This data will immediately be transferred to the CMF through a communication system, and then the MD/ACLS provider can conduct analysis, determine the best treatment, and communicate back to the EMS team on the scene using the monitor/laptop/phone/etc.

This invention also involves a system to administer medication on site, using the Pocket Crash Cart (PCC). During cardiac arrest, the most often used medications are:
Epinephrine IV/IO: 1 mg is given every 3-5 minutes to treat tachycardia, ventricular fibrillation (VF), and ventricular tachycardia (VT).
   Epinephrine (adrenaline) is a hormone produced by the human body and is responsible for peripheral vasoconstriction to concentrate the blood around the brain and the heart. Epinephrine also strengthens cardiac contractions and increases the chance of the heart returning to a normal rhythm.
Amiodarone IV/IO: the first dose is 300 mg, and after 15 minutes, the second dose is 150 mg.
   Amiodarone is another important medication usually given to slow the heart for ventricular fibrillation and ventricular tachycardia (the most common cause of cardiac arrest) if electrical defibrillation is unsuccessful.
Atropine IV/IO: 1.0 mg every 3-5 minutes is given to treat bradycardia
   Atropine blocks the effect of the Vagus nerve which slows the heartbeat and acts on the conduction system of the heart and accelerates the transmission of electrical impulses through cardiac tissue.

Other medications can be added to the Pocket Crash Cart if needed.

The manual version of the PCC is good for advanced medical settings with limited ACLS responders. The manual PCC is comprised of multi-port IV tubing with attached prefilled color-coded syringes with epinephrine, amiodarone, and atropine. An example of the amounts of each medication is explained below. These medications can be stored at room temperature.
1. epinephrine: 10 mg, which will allow for 10 bolus administrations
2. amiodarone: 450 mg, which is enough for 2 bolus administrations (first: 300 mg, second: 150 mg)
3. atropine: 3 mg, which will allow for 3 bolus administrations

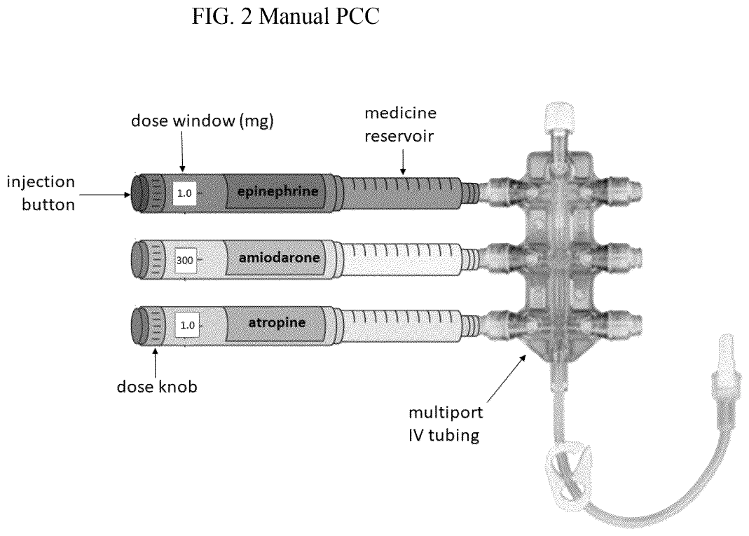

The amount and concentration of each medication can vary. The syringes are attached to the IV line with one port connected to Normal Saline 0.9% IV bag for flush (FIG. 2).

Each syringe (pen body) shown in FIG. 2 consists of a cartridge holder with the medicine, plunger, dose knob, dose window with the dose indicator, and IV connection port with seal.

This device can be suitable for pediatric use as well if the dose indicator is calibrated in 0.01 mg.

After establishing IV/OS on the patient, the PCC is attached to the IV/OC port. Depending on the cardiac heart rhythm and patient condition, the MD/ASLC provider can decide which medication(s) to administer. Then, they can administer the medication(s) directly at the scene by turning the dose indicator to reach appropriate medication dose and then pushing the knob to administer. They can announce the dose and time of administration, allowing the voice recognition software mentioned above to transcribe their speech on the computer screen. This information can be sent to the CMF for record-keeping and to continue to monitor the patient's response to the interventions.

During CA, the medication can be administrated without leaving the patient to open the currently used crash cart and manually attaching the appropriate syringe to the line. So, the current invention saves time and resources to allow first responders to resume CPR more rapidly after medication administration.

An Automatic version of this invention would allow remote administration of the aforementioned medications, such as by using a phone or phone-like device at the scene. These medications may also be remotely administered by ACLS providers at the CMF once they assess the cardiac rhythm of the patient and determine the dose necessary. They may press a button on their laptops/computers/phones, and the Automatic Pocket Crash Cart will administer the proper amount of medication.

We claim:

1. An improved Cardiac Arrest Resuscitation process that provides accurate information about the patient before admitting to the Emergency Room (ER) at the hospital based upon data accumulated from the accident site, comprising of the steps of:
a) Acquiring accident data performed by EMS team or first responder at the accident site such as patient information, brief description of accident site, and any witnesses or family information;
b) Entering said accident data into an on-site computer via video camera and speech recognition software;
c) Transferring said data from on-site computer to a Central Medical Facility (CMF) computer;
d) Analysis of said accident data by MD/ACLS provider,
e) Developing diagnosis based upon said accident data;
f) Transferring said CA diagnosis to a second computer located at the ER;
g) MD/ACLS provider sends orders to administer medication via manual PCC to the EMS team at said accident site or administers medications using APCC themselves at the appropriate time;
h) Displays results on the on-site computer monitor at the accident site for EMS team to review and continue resuscitation process.

2. The CA process of claim 1, wherein said utilizing heart rhythm to analyze extent of heart damage diagnosis.

3. The CA process of claim 1, wherein said heart rhythm diagnosis includes a visual display of EMS team to MD/ACLS provider communications.

4. The CA process of claim 1, wherein said heart rhythm diagnosis includes a real time monitoring of the accident scene.

5. The CA process of claim 1, wherein said heart rhythm diagnosis prior to admission of a patient to an ER based upon data accumulated from the accident site, comprising the steps of:
a) Acquiring accident data performed at least at the accident site and within an EMS vehicle, wherein said accident data includes accident site information, patient information, physical environment information, interventions and medication administration;
b) Entering said accident data into a first (EMS) computer;
c) Transferring said data from said first computer to a 24/7 central medical facility;
d) Analyzing said accident data;
e) Developing heart injury diagnosis based upon said accident data;
f) MD/ACLS provider send orders (to administer medication via manual PCC) to the EMS team at said accident site or administering said medication themselves at the appropriate time;
g) Displaying said results on the first computer monitor at the accident site for EMS team to review and continue resuscitation process.
h) Transferring said CA diagnosis to a second computer located at the ER; and wherein said step of analyzing said accident data based on collected information and video monitoring.

6. The CA process of claim 1, wherein said comprising:
Defibrillator equipped with video camera, voice recognition and mirroring display software, access to internet and display for notes.

7. The defibrillator of claim 6, wherein said comprising:
a) display and analyze the heart rhythm to advise for electric shock (number of Joules can be adjusted);
b) display EMS team communication and transfer the heart rhythm and acquired data from accident site to the EMS team computer;
c) EMS team computer transfer acquired data to the central medical facility for MD/ACLS provider review;
d) display and analyze the heart rhythm, synchronize for transcutaneous pacing;
e) display communication notes.

8. The CA process of claim 1, wherein said includes:
EMS team computer (laptop, phone, table, pad, etc.) which can be equipped (instead or to addition to defibrillator) with video camera, with voice recognition software, mirroring display software, access to internet and ect.:
a) Acquire data from accident site (includes accident site information, patient information, physical environment information, interventions and medication administration)
b) Transfer data to secondary computer at the central medical facility 24/7 coverage) and receive feedback from MD/ACLS provider.

9. The CA process of claim 1, wherein said comprising:
Manual Pocket Crash Cart.

10. The Manual Pocket Crash Cart of claim 9, wherein said comprising:
a) Multi-port needleless intravenous connector with integrated check valves, slide clamps tubing to be attached to the patient's IV;
b) wherein said at least three color coded syringes with emergency medications as:
c) Red colored syringe filled with Epinephrine (red for STOP tachycardia), 10 mg which will allow for 10 bolus administration);
d) Yellow colored syringe filled with Amiodarone (yellow for STOP tachycardia), 450 mg, which is enough for 2 boluses administrations; first: 300 mg and second: 150 mg),
e) Green colored syringe filled with atropine (green (for RUN increase heart rate with bradycardia), 3 mg; which will allow for 3 bolus administrations);
f) Wherein said one of the port attached to Normal Saline 0.9% IV bag or syringe for flush.

11. The Manual Pocket Crash Cart of claim 10, wherein said each syringe comprising:
a) Syringe body with dose window and dose indicator, dose knob, rubber seal and a label.
b) Inside syringe body comprising of cartridge holder and plunger.
c) To administer medication, choose appropriate syringe, turn the dose knob to select the appropriate dose depending on the heart rhythm displayed on the d defibrillator from the claim 6, push the dose knob in until it stops, and “0” is seen in the dose window.
d) Flush with 0.9% NS by opening the slide clamp.
e) Syringe body with dose window and dose indicator may be calibrated to pediatric doses to expend the use of PCC.

12. The CA process of claim 1, wherein said comprising:
Automatic Pocket Crash Cart.

13. The Automatic Pocket Crash Cart (APCC) of claim 12 description is intended to be illustrative, and not restrictive, wherein said comprising: a pump configured to deliver appropriate medication to the patient and user interface including a processor and display.
a) The case of a water-resistant enclosed apparatus with the cassette comprising of medication vials attached to the pump and delivery mechanism, battery, processor and Bluetooth, communication, USB ports.
b) The APCC wherein said allow to manually use clinical status information to entered into the APCC via the user interface to choose appropriate medication and dosage. It is record date and time and display on the screen for easy monitoring
c) The APCC of claim 13, wherein the patient's clinical status information is downloaded into CMF system via communication port.
d) The APCC of claim 13, wherein the communication port is a wireless communication port to CMF computer screen/laptop/phone etc.
e) The APCC of claim 13, wherein the user interface is part of the medical infusion pump.
f) The APCC of claim 13, wherein the user interface is part of device remote from the medical infusion pump.
g) The APCC of claim 6, wherein if the received patient clinical information aka heart rhythm indicates medical interventions, medication could be administered manually or remotely.
